SEQUENCE LISTING (13 sequences)

SEQ ID NO: 1 - DNA, 369 bp, homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 2 - protein, 122 aa, homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 3 - DNA, 570 bp, homo sapiens
agggacccgcagctcagctacagcacagatcagcaccatgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactgagcttcctcttcactctgctctcaggagacctggctatgaggccctcggggcagggatacaaagttagtgaggtctatgtccagagaagctgagatatggcatataataggcatctaataaatgcttaagaggtcaaaaaaaaaaaaaaaaaaaaaaaaaaaaaa

SEQ ID NO: 4 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGHGAEDSLADQAANKWGRSGRDPNHFRPAGLPEKY

SEQ ID NO: 5 - DNA, 4286 bp, homo sapiens
gatggttgacaactcccctcctcttccccctcttctactgtctactcctgggaccaagtgagccacgccagctcagatactacactgaccacagggaatcccaccttttccaaggaatggaagttgtgtagggaatattcaaatgttgcttagcattgccttagataagaaccaaagggacagggaaatcctctgacagctatctgccttataactttcattttactgtgcctaaaatatgctcagaacccagaaagaggcataattcctaattttggcaggctctaatctaaaataatgattctcaaacatggtgtgacttttgtctatttgctttatcctgggtcactgctcctcttctgtcagatactgggattccaatgagacaaatggaaatggagacgtagaccctctgaccttctatcttttatctatacacatacacctgtgtgtgtgtgtgtgtgtgtgtgtgtgcgtgtgtaaaaccgagtgggtttttttcttggaatgaaagaatggactaacattacaaaaaataaaaacttgaaacagaatgtgtattatccttggttgtgtttccttggccctgcagcaggatgaagctctccactggcatcattttctgctccctggtcctgggtgtcagcagccaaggatggttaacattcctcaaggcagctggccaaggtgaggtccacaggatagggggcaggaggctgcttctggctgcccccaggatgcagctgagcagaggccacatccccactgggcaaaggtgctagtgatgccacagatggatagagaaggggcatggtttttcataagcgtggttcctcatgcttttctggacagctttgacactcttctatgaggatcctccagccgaggtcgcataaggtgtgagctgcctcttttcagcaggaccatgagagagatgtggagttgaggggtgcatgttcccataataccggtggggctctactgccccctagtgggaaatctgggacagttcatgtctatgtctcctgggaagccaggaagcaggtggatcaaaagtgtgaggcgagtccatggggaagctgaacggagccaaccgtccccataaaaacaaccaagcttagctgagattttaatacgtactaggcactgtttaaatgtactaatgaattggtttccatcatttagtcctatgatgcaagcagcattatcccttaacagagaagctaacacacacacacacacacacacactaacacacacacacacacacacacacacacaaaccccaagatacgtaaagaagttccaaagcagagcaggattaacccaggcagtcttgctctgcagaacttgctcttaatcaaggtactctgctgctttcaaaacaagagtttcggatttgtgaacacatagctcatcctttatctaagaaatggcaaataggatgtggtgcctttggaaggtaagtctagctccacttatcccagtaaaacctacagtgaattaccttgatggtggttctactggggcttatatatggccaggaaactgctagcaagagaaatataccccgagggctgggcacagtggctcacacctgtaatcccagcactttgggaggctgaggtgggcagatcacctgaggtcaagagttcgagaccagcctggccaacatggcgaaatcctgtctctactaaaaatacagaaattagccgggtgtggtggcatgcgcctataatcccagcctctcgggaggctgagggagaagaattgcttgaactcaggaggcagaggttgcagtgagctgtgatcacaccactgcactccagcctaggagacagagcaagactccatctagagagacagagagagagagagagggagaaatataccccactagccataataaagtggcaaaattttgttttcagaatgcagtattttaaatttcaggtattattatttttctgagtctctgaaaaatggttttaaggatttgcttttaatcctatttacatgttcacacactcaactacaaatatctttcattccttaggttaatatttttcaaagggttgttctgggaccacttgcgtgagaatcacctggattctgggatgctttgtgaaatgaaatgaagattcccgggtccataccctaccccctgcccccaacagccacagtctcttgggacagagcctagaaatcttgcctttgctaagcacctcggtagatttttatgcacagcaaaggttgagaaccactacctcttgttttgctgctgaaagtgataaaatgtgccaggaattttggaagtacttattaagccaatctgaacatcaaggagccatttaagtcagtaactcagaggaataagtagagtaaaaatgtcataaactctcaataaaagcaatcaatttaacaccaggagtaataaatgcataaaatgaagatgagttatctaatagagaaattatataaaccatgattataactctatatttgagttcccccttttccgtaatcagttaattttctaaaaaatcttcgtcacttaattctagcttgatcagatcccttcagtccgtaactccctgctcctcatcttagtttagcccttcttttttcttatgccacctttcctaaggaccagagaagtgaaatgataatatattggccacctacaatgttctagacatcatacatgtattttctctgctcttctgcataatcactgtgaggcaggcaatactcctccatttcattggggaggacattgaggttctgaactagtgggtcagttgtcctttttctgaatttgattacccagtagtataaagctttcttaggtaactcacctttatcacttgctgactgaattctgacagatgtcagtttctaattatagcctggacattcagatgtattcaggaccaagttgtcctcactctacctacaggcatgaatttctctcattgactaggttaggagcgccatatgtctgcagcctccctcagaatcccctgtgttctcacaccagggaactgagggttccctgggtccttccaggtagaagttcattgtacaatgaaacatcccttaaggaccatttcatctcttctttaggtgcatcacacatggttaaaacaaagtaataacagaacttagaatggaatcaaacagaatgaaacttacaccaagtacaattctcattacattaacccagagaagtgaaaagtagaagaatatttatttcaagccaatataatttccaagggctttgttgaaggctgaaatcttcgggaggaaagtagtgagaagaaaactgttcattcctctattttcccagtatataattgttttgatcattttctttcctttccagggactaaagacatgtggaaagcctactctgacatgaaagaagccaattacaaaaaattcagacaaatacttccatgcttgggggaactatgatgctgtacaaagggggcttggggctgtctgggctacagaagtgatcaggtaatgcacattcctgatgttgccaggaatgagtgagcagagcttgactgccttggacagtcaggagagaggtaagctccttgcagagaagttagaggctgcagcccctcctcctcttgccctctctctgcctgtgtgcttagtgcgagggtctgagtggatggtagaagtgagtgattcctcaccctccctctctgggtgctgttcatccagcctaggggtgcccagcctggctgagtggggcagtgcccaggcagggtcattgttttcacccctccttccttggccttcctgggcttctcccagagtcctcccttggaaagcagagaatgggaaggtgggctgttgctcactggcctggtgattaatctccttgcttgcctggactacagcgatgccagagagaacgtccagagactcacaggagaccatgcagaggattcgctggctggccaggctaccaacaaatggggccagagtggcaaagaccccaatcacttccgacctgctggcctgccagagaaatactgagcttccttttcaatctgctctcaggagacctggctgtgagcccctgagggcagggacatttgttgacctacagttactgaattctatatccctagtacttgatatagaacacataaaaatgcttaataaatgcttgtgaaatccagtttgttattggaatctggaagcagaatatgacagtcttcctgggatcatgggcctgtttagtaccatagggatgaccaataaac

SEQ ID NO: 6 - DNA, 193 bp, homo sapiens
gttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtctgggct

SEQ ID NO: 7 - protein, 64 aa, homo sapiens
VFCSLVLGVSSRSFFSFLGEAFDGARDMWRAYSDMREANYIGSDKYFHARGNYDAAKRGPGGLG

SEQ ID NO: 8 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccatggtgcggaggactcgctggccgatcaggctgccaataaatggggcaggagtggcagagaccccaatcacttccgacctgctggcctgcctgagaaatactga

SEQ ID NO: 9 - protein, 122 aa, homo sapiens; identical to SEQ ID NO: 4

SEQ ID NO: 10 - DNA, 369 bp, homo sapiens
atgaagcttctcacgggcctggttttctgctccttggtcctgagtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggccgcagaagtgatcagcaatgccagagagaatatccagagactcacaggccgtggtgcggaggactcgctggccgatcaggctgccaataaatggggcaggagtggcagagaccccaatcacttccgacctgctggcctgcctgagaaatactga

SEQ ID NO: 11 - protein, 122 aa, homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 12 - DNA, 10001 bp, homo sapiens
gggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctggtaatccatagcccatggcctggctttacttctgtaaagtggcaggagaccttttgacagctgagccatttcttattttatttattttaataagagatggtaggaatgagcaatgatattagtacctggggactgttgttcttaaggagaaacaatcttagaatgattagtgataccccttgctttctcttttctttcattatactttttgtacacatatttttcccatttatttattggaatcttactgatttattataagtataagctttatgtctacacatgtataatcatttttccccaagtataagtctctttttcatggaggcacagcctagacctggttagccgccatctcccctcattgtatgcccaatatctattgtagtatctgctgcatagaaggcactcgatgcgtgaatggataatgactgatgatgaatcaataaataaatggacatgtcattgtaaaaaattctaaaaatctagaataacacaagctgttggcactacctagaaacacagatgtaaaacttcctaggttgtgtttcaccatgggaacatgtctttgaacaaaaatgggatcatattctattgcactctttcccttaagagatacttctccaggtcattaagtgctcttccacaatatcagtatatggcagaggcaaggtcataccaggtctgtctgaaaccagggcttggctcttaacttgcagccatactgcctccaagtctaggtggctgggttttaggatctgtaatgggaactcagtgtcacaacctctactgggaaggtattctggtgttgcataacaggactttctgttagagataaccatggcaaaatggaatagagacaaagttcaggtttctgctgccaggagctgagattgctgtgaccaatggcattctcccaaaccaaataatccaacctggaattaccataaaccactcctcatcttttcaaggggtgtccaagttcccagaaaagaacatttgttaagggatggaggcaaggaggtggagaagaaagagcactggccaaggtatcatgagtgtcctgggttctggtccttgaataagccatttatcttctctgcagcttctccatctgataggagtttggaggcagagttttttcttaatgagcaaaagacagtcgtgcctaggagatgtggtgtacatgttagaaagaagggactggctgtgactctataaaagatgaattcatacaaaaacaaattaccctttcccagggagaaagtttggatccagtaattagagatctcaaaaagtagaagacctgccctgtgaggcctgtggcctccaagtttgaatgctgtgtgtcagctttaaaaactagtttcttgctgataaatgtttcatattaagcatgtgttgagagtactccttgcctaccttcactagccactgtttccttcccctcctcccttgtcccttcattctctccagaactttctgctaacttccattctcttcaggacttcagcatggttgggagaagatcagaaaggcatcctcactgtttttattttagtccacttgacctttggggagtagttccactggctcataagtatcagccccccatagcacagcaccccacactgagcccggaagcaataaagaatcccaatctgctgtcactaaccagcacgctcaactgccatgccctttactcttctcatctccctgctttcacgtcacaccaactaatttctctatgagtcagcctcaactctcccaacactctgcccacccttcttctactaccttccagtgagctcctcgaaagaagggtctgcggtgaggatgcccctttatctctgcctatttccttcccattacaaaaacttgaaacctgcctttcccatgttgatttcactttattctcatctttacccatggggtatgcctcctgcaattcctcctagacaatagaatgagaaagaggggtcctcgtcctctttgctttccatgaccatttctccattcttcacctctgtgatgtgtcctctttgaagtccctgataaattcattaccaccttctctccagtcttactaatgttatctgcacaagtgatttccaaacaggaagattttcaaacactgattcctgaagatcacccccaactcgctgaactgagaccaagacctccaagattatggcttaggaatctgcattttttttttttttttgagacaagagtctcgctctgttgccaggctagagtgcaatggtggaatcatagctcattgtaacctcaaactcctgggctcaagtgatcttcctgcctcagcctcccaagtagtgaggacaacaggagtgtgccaccatgcccagctaattgttaattttttgtagaaatggagtctcactatgttgctcgggctggtctcaaactcctgaccttaacccatcctccgcctccgcccccaaaagtgttgggattacaggtgtgagccaccgtgcccagcctagaaatacccactagaagcttctgtgtagacaatctgcttagtgatgtttggagacaaagtacctctttattgtattcattgacaaaactctccagtcctctcccatcttcatggaaaattttcacagttcatttacggccctctttccaacacattcactgccaatactcttattgacaataactgtattgttgaaccttccagtatcctgcattcccggatcaaggccccctcaaagccctgatatgcaaatatctgggaaaagaatgttccagaggaaaggaacagctaatccgaggcccctagggtaagatgtgcctgggggtttggagaccagtgtggccagagcaaaatgagcaggaggagagaattggatgatgaggtacgagaggaaggagttaggacagtttgagtaaagtttgaaaaccattataagggctttgacttcaactatgagtggaagtggaatcctccggagagttttgaatggagagtgatagaagttgtcttgtgttgtaacagtctggctgctatactgaaaagagactagttggcggcaaagggggaaatgtggaagccagttaagaagccatcataacccagaaggtgatgcctaataacatctctctgggagcagcggagagatgataagggtttgccttctgaatatgttttttgacaattaatgtaaacatttcaagtaggctgagattttattgcatattaacaatgtccatgttcactcgcggcagccgcccccttctgcgcggtcatgccgagccagcacctgggcctggaactgggccgcagcccccagcttcacccaccacctccctaccatggacccctgcaaagtgaacgagcttcgggcctttgtgaaaatgtgtaagcaggatccgagcgttctgcacaccgaggaaatgcgcttcctgagagagtgggtggagagcatgggaggtaaagtaccacctgctactcagaaggctaaatcagaagaaaataccaaggaagaaaaacctgatagtaagaaggtggaggaagacttaaaggcagacgaaccatcaactgaggaaagtgatctagaaattgataaagaaggtgtgattgaaccagacactgatgctcctcaagaaatgggagatgaaaatgtggagataacggaggagatgatggatcaggcaaatgataaaaaagtggctgctattgaagtcctaaatgatggtgaactccagaaagccattgacttattcacagatgccatcaagctgaatcctcgcttggccattttgtatgcaaagagggccagtgtcttcgtcaaattacagaagccaaatgctgccatccaagactgtgacagagccattgaaataaatcctgattcagctcagccttacaagtggcgggggaaagcacacagacttctaggccactgggaagaagcagcccatgatcttgcctttgcctgtaaattggattatgatgaagatgctagtgcaatgctgaaagaagttcaacctagggcacagaaaattgcagaacattggagaaagtatgagcgaaaacatgaagagcgagagatcaaagaaagaatagaacgagttaagaaggctcaagaagagcaggagagagcccagagggaggaagaagccagacgacagtcaggagctcactatggcccttttccaggtggctttcctggtggaatgcctggtaattttcccggaggaatgcctggaatgggaggggacatgcctggaatggccggaatgcctggactcaatgaaattcttagtgatccagaggctcttgcagccatgcaggatccagaagttatggtggccttccaggatgtggctcagaacccagcaaatatgtcaaaataccagagcaacccaaaggttatgaatctcatcagtaaattgtcagccaaatttggaggtcaagcataatgcccttctgataaataaagccctgctgaaggaaaagcaacctagatcaccttatggatgtcgcaataatacaaaccaacgtacctctgaccttctcatcaagagagctggggtgctttgaagataatccctacccctctcccccaaatgcagctgaagcattttacagtggtttgccattagggtattcattcagataatgttttcctactaggaattacaaactttaaacactttttaaatcttcaaatatttaaaacaaatttaaagggtctgttaattcttatatttttctttactaatcattgtggatttttccttaaattattgggcagggaatatacttatttatggaagattactgctctaatttgagtgaaataaaagttattagtgcgaggcaaacataaaaaaaaaaagtccatgttcatctctaaatgacatcattgttccaaagcttttccattcttcttaaccttccacctgtcaatctataggagatgacttctcctacttcactcatgcattgactccttcaatcaataaaagtgactaagaacctgctacaggtgaggtgctgtgtttggtgttaaagtgacaacagttatctgtcaataagcctgacaaggttcctatccctgtgttttgtgcactctgggtcaaactcagaaatgcaaacaggtggagagcgatgagttctatgactggtaaagaaaagggcctgctggtttccctcaggatctctgtccttcatctcaaaatgcatcttccttgttatcgttcctctccttcctgtctcagaggaagacctgctcctgctacactctgggcaaccttgtccccgtggccctgtggccccttggttgttgaagtctatgttatgccctatcttttaccctcagtcactctctctgttaacattctccctgtgccctgtaaccctccctcatctttaaataaatcctcctcctttgaccttcgcatgtattcagtcatgcaactcaacaagcatttattgcacagtgatattcaatttgccacttgctaaaagtctgaaccttggcagctgaatgtgatcagaaaaaaagcacgactgctatgactagtctcactttaaattcatggtcgttgaccaagagctaccatacaatccactacctttctcaagttcagtcacattcttcctttcctagatgtctgctttctacttctcttctcttctgaaacttcccacaactcctcgttcattctcttctcagttgacaactttgcttcctatttcactgaaaaatagaagcaatcagatatgaacttctggctgggcatggtagctcatgcctataatctcagcactttgggaggccaaggcaggaggactgcaggttaggaatttgagaccagcctgggcaacatggtgaaactcccactgtactaaaaattttaaaaattactcaaacatattggcaaacaactgcagtcccagctacttgggaggttgagatgcaaggatcacttaaacctgggaggctgaggctgcagtgagccatgattgcaccactgcactccagctcaggcaacagagcaagaccctgtcttgagaggagaggagaagagaggaggggaggggagggcaggggaggggaggggaggggaagggagaggggaggggagaggggaggagagaggggaggggaggggaggggaggggaggggaggagaggaggatcaggtgaggagtatgccaaggagtgtttttaagacttactgttttctctttcccaacaagattgtcatttcctttaaaaagtagttatcctgaggcctatattcatagcattctgaaagaaagaaaagaaaagaggaaagaaagagagaggaaggaaggaaggagaaagagagaggaaggaaggagaaagagagaggaaggaagggaggaagagaagaagggaggaagaaaagaaggaaggaaggagggagggagggaagggagggagggaaagaggaagaaaggagggaaagaaggaaggaagagagagaggaaggaaggaggaagagagaagaaggaaggaggaagacagagagggagtaaggaaggaaggaaggagaaagagagaggaaggaagaaatgaaggaaggaaggaaagaaagaaaaaataaaagagtgaaaacggactggagaagaagaaaccacagttgctgctatatccaccagcctctctgcatgtcctggcctcagccctgctgggctctggtactgaccacttccttccttcctaatttcctaattgactaggccagctgagcagggcttttctgtgctgaggaggtaaatctctggatatctagactgaggggtggaaggagccttccagggcacacatgagacatggcaggggtaggctgctagttttattttgttttcttttagacacagggtcttgctctgttaaccaggctggagtgcagtggcgtgattatagctcactgcagccttgacctcctgggtctcccacaatccttccgcttcagcctcttgagtagctgggactgcaggtgcacactaccacacccggtccatttatttttatatttcgtagagacaagatcttacagttttgcacagagtgatcttaaactcttgaccccaagtgatcctcctgccttggcctccaaaagcattgggattataggagtgagccactgtgctggacctagtctgtcagctttgaagctttagatatgaactcagagggacttcatttcagaggcatctgccatgtggcccagcagagcccatcctgaggaaatgactggtagagtcaggagctggcttcaaagctgccctcacttcacaccttccagcagcccaggtgccgccatcacggggctcccactctcaactccgcagcctcagccccctcaatgctgaggagcagagctggtctcctgccctgacagctgccaggcacatcttgttccctcaggttgcacaactgggataaatgacccgggatgaagaaaccactggcatccaggaacttgtcttagaccgttttgtaggggaaatgacctgcagggactttccccagggaccacatccagcttttcttcgctcccaagaaaccagcagggaaggctcagtataaatagcagccaccgctccctggcaggc

SEQ ID NO: 13 - DNA, 10001 bp, homo sapiens
gtctgccagggagaggtggctgctatttatagtgagccttgctggtctcttgggagggaagaaaagctggatgtggtccctggggaaagtccctgcaggtcatttcccctacaaactggtctaagacaagttcctggatgccggtggtttcttcatcccgggtcatttatcccagttgtgtaacctatgggaacaagagaggtttgctgtgccttggcaatggacagggtgctagatcagctctgctcctcagcattgggggaagtgcagctgcagagatgccagtgggagccccgtgatggcggcacctgggctgctggaaggtgtggagtgagggcagctcttcagccagctcctgactataccggtcatttcctcaggatgggccctgctgggccacatggcagatgaccctgactgaaatccctgtgagttcatgtctaaagctttaagctttaaaacggacagcctacccctgccacatctcatgtgtgccctggaagcctccttccacccctctggatgtcctgatatttctcagcacagaaaatctctgctccgctggcttagccaatttggaaatgctttttctaagttggctcctgagccaaggacaatgtagagagggggactttctgctgccccagcctagtcctggagccccaccttgggagaatgagagtgtggtgcgttaaataggcagcccagctggggacgtgcccagcatccaggcagggaagggtgggagagctcttggtctgctgtattatcacggaggggtgcagggggcatgcagatcactctctcatgagaacatcaacagggtcagattagctctgcagaggcttatggaggagcatggtggccagagatgggtcagtaccagagcccaggggggctgaggccaggacatgcagagaggctggtggacatagcagcaactctggtttcttcttctccagtccatgttcataccctgagggctaggcatttgtaataacaaacaaacaagcaatttagaaatgggccaggcatggtggcatgtgcctatagtcccagctacttgggaggccaaggcaggaggcctgcttgaacccagaaatttgaggccagcctgggcaacacagcaagattatcttaaaaaattttttttaatctctgagaaatgggtagggccaggaagtaaaggatggccaaatactccataagcagcaaatgcgtggctccaatgtgaacaatgatattatagactctgttctgagacctatgcattgacacctccacctcccccactacatcttgccaccttaaaaccactgagagtggtacctgctggaatgggtccacacacacagtcacacatattttaggcagggtagttgacatccccagggaaaaagagctcacagagagaggctgaatgtttccaactgggtagcagtaatagtacatcatgctgtacatggtacagcacagatcaggtgaaaataatagcacatcgtgattaaccagggcttattccagggagtcaagaagagtttcatatcagaaaaatctatctttgtaattcactataccagtaatcaaagaaaaggattgtacatttattttactagatgcagaaaatgaatttcataattgtcaacatctactgatgataaggaaaatgtataacaaaataaagagaccatttctgacttgagaaaggataaataccaatatgttatagcaacagttctcaaactgttttccagggaaccctaagaatccctccttagggaggctttgatctcaaaattatttttagaatagtgctaacacactattttcatgtttcagtctcattttctcatgagtacacacaatatgacaagttagttgatatgagtgtggatttccacatggtaactgacttttcagaagctaccacttgttgagtttggtataatatagaatagccacaattatctaaaaataccattaaaatacactcccccatttcaactatatatctgtgtgaggctgaattttcttcatatactccaacctaaataacatattaaaacaggttggatgatgaatcagataggaaaatccagctatgaaaaaaaaatcagacatgaaaaattttcaaaagggtaaaaccatagtactcttcttactttttttcttttggaagatggttatttttcataaaaatatattatttatgttaacatatagaagatggataattttttgaagaattgataaatgtttaaattttttctttctattatggtaaatactgatgaatagagtccccataaataaaagttctttggggtattcaataatttttaatagtgtaatgggatcctgagaccaaaaggtttgagaatcattgctctacagcaaacattatgtgtaattaagacacttcaggtgcattctcaagaagaccaataaagaggccacaatggcaggcgtggtggctcacacttgtaatccaagaacttagagaggacgaggcaggtggatcactggaggtcaggaattctcaaccagcctggccaacatggtgaaaccctgtctctactaaaagtacaaaaattagtcgggtgtagtggcaggtacctgtaatcccaagtacttggggggttgaggcaggagaatcacttgaagccgggaggtggaggctgcagtgagccgagatcgtgccactgcactccagcctgggcaacggagtgagacttcatcatggaaaaaaaaacaaagaggccaggatgtctggttgttactgccactgtttcacatatccctgaaggacctgcccaatgctaaagaaacacaaggaaggtaagaggtgaaagagaagaaatgaaactatcattgtttgaagatgacaccatcttttacatagaaaacctgttagaatcaaatggcaagctattagaactactaagagaattcagtgaggctgctgtattcatggcaaaattttaacaattgatagcatttctctgcaacattccttaatagttataaaatacagcacaaagtagtaccaaaaatattaactatctaggaaataacctcttacagagaaaatttagtctgttaaaggataaacagtggcaatgtacgtcatgtccacagagattatattttagcttagcaaagataccaattctcccaaatttatttataaattaaatgcaatgtgaatcaaaatttcccactggaatttttatcaggaaggcaacaaattctttctttctttctttctttctttctttatttatttatttatttatttatttatttatttccttccttccttccttccttccttccttcctttctttctttctttctttctttctttctttctttctctctctctttctctctccccccctctctctctctctgtctctctctctctctctttctttctttctttctttctttctttttaagacaaagtctggctctgtcacccaggctgcagtgcagtgatacaatctcagctcactgaaacctcaacctctccggcatcaggtgaacctcccacctcagccccccgagtagctgggactacaggtgcacaccactgggcctagataactttttgtatttattgtaaataaacacaaaaaataaatattttgctcaggttggtctggaactcctgggctcaagcaatccgcctgccttggcctcccaaagtgctagaattacagttgtgagccaccacacccagccaataaattaattctttatgatgaataagttatctatgaaaattaagtcagctgggtgcggtggctcacgcctgtaatcccagcactttgccgggctgaagcaggtggatcacctgaggttgggagttcaagaccagccggaccaacatagagaaaacccgtctctactaaaaatgcaaaattagctgggtgtggtggcatatgcctgtaatcccagatacttaggaggctgaggcaggagaattgcttgaacccgggcggtggaggttgcggtgagccaagattgcaccattgcactccagcctgggccacaagagcgaaactccatctcaaaaaaaaaaaaagagaagttaagtcaatgaaaagttaagtcaattaaaaaagtaagagctgtagtgtttagatatatacacacacacatatatatatatttatctttatatatgtatatatatcttttcctttttttgagaccgagtctgtttttgttgcccaggctggaatgcagtggcgcgatctctgcttactgcaacctctgcctcccaggttcaagcgattctcgtgcctcagcctcccgagtagctgggattacaggtgcctgcccccatgcccggctaatttttgcatttttagtagagacggggtttcaccatgttggccaggctggtctcaaactcctgacctcaggtgatccaccggcctcagcctcccaaagtgctgggattacaggtgtgagccaccgcgcccagccatatattttgcttttcatctgcagctcctggatcctaactccttgttatattgttgggcactttaggcctcagtaaacagaatctctgtctatgaccttctcctgtccttcttccacctgcccaaagcaggactctaatttgattgtgggtcaaaagactctcattccagaaagggccttgcctcataccctagaggaaggaatgctgcacagaaacgccaagtctgaacagacaagccttgctgggtttataccatatgctttttgtccaatcacatttcttcatggttgccaatcatgcctatgtaatgaagcctccataagaacccagaaggacagggttcagagagtttccacatagctgaacactatctggagagtgaacacttcctagagagtggcacacccagagagatcatgaaagctccacgcccctttccccttacctcgccctccacatctcttcatctgtatctttcataatatcctttataaataaaccagcaaatgtgtttccctgagttatgtgagtcactctagcaaattaatcgaacccaaagagggggtcatgggaaccccaacttgaagccagtcagtcagaagttccagaggcccagacttgcaactggggagaaagagggggaggtcttggggactgagcccccaacctgtgggatctgacactgtctccaggtaggtagtgttggaactgcattggaggacactcctggtgtctgctgcttggtgtgtggggggaaaaacccacacctttggttacggaggtcttctgtgttgacgatcattgctgtttgagggcagagggaatacacggtttgagagagtttttccctgacatgagcgaacaggggacatgtactggtctctgagatgggggatcatgggatctgccacaagtggggagaccactgtgacccctgccacagtctttggggcagagggtgtctcgggggcagaagaagcgagagttgtttgcagtagcagttatgtccaaagtgggcgccaggaaagtagggctgcccagctttgaagagcctccttactcccagcctgaatgaaaccatttcctgtaaagcgctaagcataaagtttgccaatggtgatccacggagaagtgagtgtaccccaccccgccatcccacagggaatgtcggagtgatgttgatctgcacctagggaaggaatggttcatgagatgtggtggagatgctgagggcccgtggacatcagatcctaccctacctgtgccaggacaagccatgcgcatgtgcttcagaccaccaggcaacaggagtgttgcatgaggtgtgaagcaggcacctgggaaagaggagtgtgaacagcagatgggacacactgggggcagtcataggaatgaaatgtcccaggatggatgcaggcaggttatggaggacttagtgaggactgctctcctggtgggaattgtggagtgggagactggatggagactggaggtgttttaagtagggaagccaacttgcaagggtgaccagggaaactatgtcggccaagggtgagacatgcactggcaagactctcagacagcctggcttatctaagcagaatgcttgagccatgccaacggtgcctcgcaagttgtattaatcatgtcctttcattttgtgtttttggtgcttggcatctgggcccttgctgaccctaagggaccatttctctcagagctagtcaagtcctagacacagtaaatgactctcctgggagcatgccttccatgtgcagaccaaccaatcaagagtccacactcccacccacctcctttatcgagctctcacatcctggggcaccatccacctgccctaatcactcaaggaccacgtcccaaacaactagggacagcctccatgcccctgcacccattgaaattattcatgctagccaatcctaaacctgtgtatgctgccacaccattccttcctgcagaaacacagtaaggactcttcctacacctcccctacttcctctgctccctgacttacccacttacttcctggtgcagtcccctgtggcatagttcactctcttcttttgggaactgtgaggctatcttctcaatggcagtcatctcctgagctgttggccttgccatacctaactaataataaaatctatattctaaggtaaaaacaaaacagatagggtctcactctgttgcccaggctggagtacagtggtgtgatcatgactcactgcagcctcaaactcctgggctcaagcagttctctcatctcaacctcccgagtagctgggactacaggcacacaccaccatgcctggctagttttcttattttttttgtagatacagggtcttgttatgttgccaaggctggtcttgaactcctgggctcaagtgatcctcctgccttggcctcccaaactgctgcaattacaggcatgagccaccatgcccagatcagaaatcttactaaaaatatttcaaggagaagagaaagccaaagatgttgaatatatatatatgtgtgtgtgtgtgtgtgtatatatatgtatatatgtgtatatatgtgtgtatatatatatgtatatatgtatatatatatgtatatatgtatatatatatgtatattggggcaggcgtggtggctcatgcctgtggtcctaactacttgagagtctgaggtgggaggattgcttgagcctgggagatcgaggctgctgtgagctgagactacaccactgcactccagcttgggtgacagagtgagaccctgtctccaaaaaaacaaaaagaaaaagaaaaaaagatggaaaaagacatgaaaaaacaacaacagaaatacccacacatcatcaatgggagggaagcatcttgaggcagcaaagcgggagtgctagtagagaggcagatagggcgttggacctgaggcattaaggaaagtcaggatttggagcttacaagtctctcattggagatgggatggggttggaatgaatgtctgagcaaacacaaagcatttccttccctaatgactccccaccagtctaaagaatcccacattaggtcgaacacggtggctcacgcctgtaatcccagcactttgggaggccaaggcgggtggatcacgaggtcaggagatcgagaccatcttggctaacatggtgaaaccccgtctctactaaaaatacaaaaaaattagccgggcgtcatggtgggcgcctgtagtcccagctactcgggaggctgaggcaggagaatggtgtgaacccgggaggcagaacttgcagtgagcctagatcgcgccactgcactccagcctgggggacaaaacgagactctgtctcaaaaaaaaaaaaaaaaattcccacattagagttggggaaatgggcagtcctggtggaagttagggaacagatctgggacacgttatagccagctggactacaggaggccataagctcaattcttccttgactctgaaaccttccactggtcctaatgcctagtaattccaggcctttcccagttgtgccaggcttggaggtgaacacatctatgtgccaagaaggaaaggtatgccaagcaggggcttaagtcatccttatcctcagtctgtctatgagtggtatgtacccctgttccccttgcaagatctgctgggcttaggtctcctggctgtgagttccccatacctgggcataaatgtagtgagcctgagctcccaaataaggttgggggctccagagaggtggagagccctgtgtctgggaagtgtgcccacccagcaggtctgaccaggaagatacactgctagggttatggaaaaagactatgtgtcaaggtctcttgattctccatctaggcagagaatcatctttaattaatgggaaactggaaggcaaattacttggacctgaaattactttttgtttattgaaccactgtgttgtaaatcacatctctctgaaggcaagagaaatcagggagttacaaaatgtttaggagaactaaacaggactccctgttttgctaactaatcagattgagacaggctctctggtaaatctacaaatttgatgttgttcaaccataagcagtaaatttcctatgctggattttcctgacaatgaatgtaaaaggaaaaggagtctttttgacaaaatattttattgttcatctaaactgaaaaacttctctatttttcaaaattgctatacgtgtttaaagatgtagatatttgaatagcctaactggtacagaaggtttaatgatgattcctaagacatacctataaattacttgaaattgaaacgaaatttaagaagaattattggaattttccccttctcaaatgagttcttagtttcataaatactatacaagtccataagagatttggggttttgagatgtctttttttttttttttttttcagacggagtttcactgttgttgcctaggctggagtgcaatggcgtgacctcagctcactacaacctccacctcccaggttcaagcgattttcctgcctcagcctcccaagtagctgggattacagggacctgccacaacgccaagctaatgttttgtatttttagtagagatggggttcaccatgttggccaggcttgtctggaactcctgacctcaggtgatccacccgcctataatttattactcccttttgcaaatgtttgaaaaggaataaagtgcaatatttttaaacagaatgcagagttctgttgtcctttggcaataccagtttcagactctgagagtggctcttgctgttgccgacagtgggctgatgaccaaatcccaacatgcccccgctgcgagtccttcataacctgattcagtcatcacttagaggccagcaggcttcagggaggcgtgagcctcagccaacaacctataggggaagagacgcagaactcaatgcagacaggtttggattctggtgcctagagaatgcaacttggaaactctgagccaggagaaaagggttctctctccatgagagagtgtgggctttgtgagaagcgacacacagcaaacacaattaagagtccacccctcagcggggcgcaggggctcacgcctgtaatcccagcactttgggaggccgaggcgggtggatcacgaggtcaggagatcaagaccatcctggctaacacagtgaaaccctgtctctactaaaaatacaaaaaaattagccgggcgtggtggcgggcgcctgtggtcccagctactcgggaggctgaggcaggagaatggtgtgaacccgggaggtggagcttgcagtgagccgagatcgcgccactgcactccagcctgggcgacagagcgagactccatctcaaaaaaaaaaagaaaaagaaaaagaaaaagagtccgcccctgaattaaatagttggtccttttgtgttcctggtgattcacttgctaagtggaagaaacaggagggaatcttttctcctgccctcctg

(19) United States
(12) Patent Application Publication
(10) Pub. No.: US 2006/0121039 A1
(43) Pub. Date: Jun. 8, 2006

(54) Use of agents that prevent the generation of amyloid-like proteins and/or drusen, and/or use of agents that promote sequestration and/or degradation of, and/or prevent the neurotoxic effects of such proteins in the treatment of macular degeneration

(75) Inventor: Najam A. Sharif, Keller, TX (US)

Correspondence Address: Teresa J. Schultz;Alcon Research, Ltd., Mail Code Q-148, 6201 S. Freeway, Fort Worth, TX, 76134-2099, US

(73) Assignee: Alcon, Inc.

(21) Appl. No.: 11/006,161

(22) Filed: Dec. 7, 2004

(51) Int. Cl. A61K 48/00; A61K 39/395; A61K 38/17; A61K 31/737; A61K 31/401; A61K 31/366; A61K 31/137; A61K 31/655

(52) U.S. Cl. 424/146.1; 514/44; 514/12; 514/423; 514/460; 514/548; 514/649; 514/54; 514/150

(57) ABSTRACT

The present invention provides compositions and methods for treating age-related macular degeneration (AMD). More specifically, the methods of the invention target amyloid proteins and drusen that tend to accumulate in the eyes of those patients suffering from AMD. AMD is treated in the methods of the invention by providing agents that sequester and/or degrade such amyloid deposits and/or drusen such that a patient's vision is improved or restored.

### BACKGROUND OF THE INVENTION

1. Field of the Invention

The present invention relates to the field of treatment of macular degeneration. More particularly, the present invention relates to the treatment of macular degeneration by administering to a patient suffering therefrom an amount of a compound that promotes the sequestration or degradation of amyloid proteins and/or prevents the generation of and/or prevents the neurotoxic effects of amyloid, amyloid-like proteins and/or drusen.

2. Description of the Related Art

There are a number of ocular conditions that are caused by, or aggravated by, damage to the optic nerve head, degeneration of ocular tissues, and/or elevated intraocular pressure.

Just as deposition and accumulation of amyloid or amyloid-like deposits in the TM is thought to contribute to glaucomatous conditions (See U.S. application Ser. No. 60/530,430), similar deposition and accumulation of drusen (mixture of numerous proteins and lipids some cross-linked in an abnormal plaque-like fashion akin to amyloid plaques in Alzheimer's disease) on the Bruch's membrane in the retina may be a major risk factor in the etiology of age-related macular degeneration (ARMD). A number of clinical conditions, including Alzheimer's disease, exhibit abnormal amyloid deposits in tissues associated with the disease. These amyloids are molecularly heterogeneous and encoded by different amyloid genes. Amyloid protein deposition like that of drusen and amyloid in age-related macular degeneration (ARMD) has been found in various ocular tissues including the vitreous, retina, choroid, iris, lens, and trabecular meshwork in primary primary systemic amyloidosis patients (Schwartz et al. 1982). Similarly, Ermilov et al. (1993) reported that in 478 eyes of 313 patients (aged 25 years to 90 years) with cataracts, glaucoma, and/or diabetes mellitus, 66 (14%) of the eyes contained amyloid-pseudoexfoliative amyloid (PEA) proteins. Krasnov et al. (1996) reported that 44.4% of 115 patients with open-angle glaucoma also exhibited extracellular depositions of amyloid proteins. Finally, amyloidosis was revealed in the sclera in 82% of the cases and in the iris in 70% of the cases.

To date, more than 100 genes have been mapped or cloned that may be associated with retinal degeneration. The pathogenesis of retinal degenerative diseases such as age-related macular degeneration (ARMD) and retinitis pigmentosa (RP) is multifaceted and can be triggered by environmental factors in those who are genetically predisposed. One such environmental factor, light exposure, has been identified as a contributing factor to the progression of retinal degenerative disorders such as ARMD (Young 1988). Photo-oxidative stress leading to light damage to retinal cells has been shown to be a useful model for studying retinal degenerative diseases for the following reasons: damage is primarily to the photoreceptors and retinal pigment epithelium (RPE) of the outer retina (Noell et al. 1966; Bressler et al. 1988; Curcio et al. 1996); they share a common mechanism of cell death, apoptosis (Ge-Zhi et al. 1996; Abler et al. 1996); light has been implicated as an environmental risk factor for progression of ARMD and RP (Taylor et al. 1992; Naash et al. 1996); and therapeutic interventions which inhibit photo-oxidative injury have also been shown to be effective in animal models of neurodegenerative retinal disease (LaVail et al. 1992; Fakforovich et al. 1990).

To date, there are no approved effective therapies for the treatment of ocular neovascular diseases which do not include the destruction of healthy viable tissue. There are certainly no therapies specifically directed at eliminating or inhibiting the deposition and accumulation of amyloid proteins, drusen or amyloid-like proteins on the Bruch's membrane in the retina as in ARMD. Such accumulation of amyloid and/or drusen causes retinal dysfunction by several mechanisms including disruption of retinal pigmented epithelial (RPE) cell function due to thickening of Bruch's membrane, and RPE detachment resulting in rapid loss of visual acuity followed by macular atrophy and retinal detachment (Ciulla et al. 1998). Additionally, the deposited drusen and/or amyloid proteins could exert direct neurotoxic effects on the RPE cells and neighboring cells in the retina akin to the well known toxic effects of such amyloid proteins and amyloid/lipid complexes observed in brain cell death as in Alzheimer's disease (Lambert et al. 1998; Liu and Schubert, 1997; Pike et al. 1993; Nakagami et al. 2002) in retina (Jen et al. 1998). Although panretinal photocoagulation is the current medical practice for the treatment of diabetic retinopathy and ARMD and is effective in inhibiting retinal neovascularization, this procedure destroys healthy peripheral retinal tissue. This destruction of healthy tissue is decreases the retinal metabolic demand and thereby reduces retinal ischemia driven neovascularization. Photodynamic therapy (PDT) is a procedure in which a photoactivatable dye is given systemically followed by laser activation of the dye in the eye at the site of new blood vessel formation (Asrani & Zeimer 1995; Asrani et al. 1997; Husain et al. 1997; Lin et al. 1994). The photoactivated drug generates free oxygen radicals which seal the newly formed blood vessels and thereby prevent or reduce their growth, at least temporarily. This procedure has been used in patients with the exudative form of macular degeneration and many patients show regression of their subretinal neovascular membranes. Unfortunately, it appears that the PDT-induced inhibition of retinal neovascularization is risky, expensive and provides transient and temporary relief lasting only 6-12 weeks (Gragoudas et al. 1997; Sickenberg et al. 1997; Thomas et al. 1998.)

Thus, there is an urgent need for therapeutic methods for altering (by inhibiting or even reversing) the disease processes of ARMD.

### SUMMARY OF THE INVENTION

The present invention overcomes these and other drawbacks of the prior art by providing compositions and methods for treating ARMD by sequestering and/or degrading amyloid proteins and/or drusen in ocular tissue at the back of the eye, specifically the Bruch's membrane, outer retina, macula and sub-retinal space. In addition, compositions and methods to prevent the generation of amyloid, amyloid-like proteins and drusen and/or to prevent the neurotoxic effects of such proteins are provided to treat ARMD. In one aspect, the present invention provides a method for treating ARMD by administering to a patient in need thereof a therapeutically effective amount of a composition comprising an agent that sequesters amyloid proteins in ocular tissue and/or an agent that degrades amyloid proteins in ocular tissue. The sequestration and/or degradation modulates the expression of the amyloid proteins, such that the patient's condition is treated. In addition, agents that stop or reduce the initial production of the amyloid proteins and drusen, and/or prevent the nerve cell death due to the presence of amyloid proteins and drusen would also be useful to treat the patient's ARMD condition. In preferred embodiments, the agent will be a small organic molecule, antibody, protein, peptide, peptidomimetic, or nucleic acid.

Preferably, the agents for use in the compositions and methods of the present invention will mainly be small organic molecules including the following:

Compounds that may be useful for preventing the production of amyloid, amyloid-like proteins and/or drusen include: γ-secretase inhibitors such as talsaclidine (Hock et al. 2003), Xanomeline, L-689660, L-685458, McN-A-343, CDD-0097, fenchylamine, MG132, WPE-111-31C, MW-11-36C/26A, MW-167, CM-265, lactacystin, DNPS1 and DAPT (Lanz et al. 2003). Other compounds of use may include the statin family, e.g. pravastatin, atorvastatin (see Bums and Duff 2003) and presenilinase inhibitors such as pepstatin A (Xia 2003) and talsaclidine (Hock et al. 2003).

Compounds that may be useful for promoting degradation of amyloid, drusen and related proteins include glycoaminoglycans and congo red (J. Neurochem. 70: 292-298 [1998]).

Compounds that may be useful for promoting sequestration or clearance of amyloid, drusen and related proteins include gelsolin and ganglioside GM1 (Matsuoka et al. 2003). In addition, antibodies raised against drusen, and/or amyloid proteins and/or against amyloid-like proteins would be useful for sequestration and clearance of the former detrimental proteins as has been shown in the brain (Schenk et al. 1999; Janus et al. 2000; Morgan et al. 2000).

Compounds that may be useful for preventing or diminishing the neurotoxic effects of amyloid, drusen and related proteins include RS-0466 (Nakagami et al. 2002c; Nakagami et al. 2002b), V-type ATPase inhibitors (bafilomycin and concanamycin; Kane is et al. 1999), tachykinin peptides and their non-peptide analogs (Yankner et al. 1990), α-lipoic acid (Zhang et al. 2001), propentofylline (Koriyama et al. 2003), glycogen synthase kinase-3β (GSK-3β) inhibitors (Eldar-Finkelman 2002; Caricasole et al. 2003), memantine (Frankiewicz and Parsons 1999), mixed cyclin-dependent kinase-GSK3β inhibitors (Damiens et al. 2001), COX-2 inhibitors (Xiang et al. 2002) and propentofylline (Koriyama et al. 2003).

### DETAILED DESCRIPTION PREFERRED EMBODIMENTS

Human SAA comprises a number of small, differentially expressed apolipoproteins encoded by genes localized on the short arm of chromosome 11. There are four isoforms of SAAs. SAA1 (SEQ ID NO:2), encoded by SEQ ID NO:1, and SAA2 (SEQ ID NO:4), encoded by SEQ ID NO:3, are known as acute phase reactants, like C-reactive protein, that is, they are dramatically upregulated by proinflammatory cytokines. The 5′UTR promoter regions of SAA1 and SAA2 genes are also provided (SEQ ID NO:12 and SEQ ID NO:13, respectively). SAA3 (SEQ ID NO:5) is a pseudogene and SAA4 (SEQ ID NO:6) is a low level constitutively expressed gene encoding consitutive SAA (SEQ ID NO:7). SAA2 has two isoforms, SAA2α (SEQ ID NO:9) and SAA2β (SEQ ID NO:11), which differ by only one amino acid. SAA1 and SAA2 proteins are 93.5% identical at the amino acid level (SEQ ID NO:2 and SEQ ID NO:4, respectively) and these genes are 96.7% identical at the nucleotide level (SEQ ID NO: 1 and SEQ ID NO:3, respectively).

SAA is an acute-phase reactant whose level in the blood is elevated approximately 1000-fold as part of the body's responses to various injuries, including trauma, infection, inflammation, and neoplasia. As an acute-phase reactant, the liver has been considered to be the primary site of expression. However, extrahepatic SAA expression was described initially in mouse tissues, and later in cells of human atherosclerotic lesions (O'Hara et al. 2000). Subsequently, SAA mRNA was found widely expressed in many histologically normal human tissues. Localized expression was noted in a variety of tissues, including breast, stomach, small and large intestine, prostate, lung, pancreas, kidney, tonsil, thyroid, pituitary, placenta, skin epidermis, and brain neurons. Expression was also observed in lymphocytes, plasma cells, and endothelial cells. SAA protein expression co-localized with SAA mRNA expression has also been reported in histologically normal human extrahepatic tissues. (Liang et al. 1997; Urieli-Shoval et al. 1998).

SAA isoforms are apolipoproteins that become a major component of high-density lipoprotein (HDL) in the blood plasma of mammals and displaces A-I (ApoA-I) and phospholipid from the HDL particles (Miida et al. 1999). SAA binds cholesterol and may serve as a transient cholesterol-binding protein. In addition, over-expression of SAAl or SAA2 leads to the formation of linear fibrils in amyloid deposits, which can lead to pathogenesis (Uhlar and Whitehead 1999; Liang et al. 1997). SAA plays an important role in infections, inflammation, and in the stimulation of tissue repair. SAA concentration may increase up to 1000-fold following inflammation, infection, necrosis, and decline rapidly following recovery. Thus, serum SAA concentration is considered to be a useful marker with which to monitor inflammatory disease activity. Hepatic biosynthesis of SAA is up-regulated by pro-inflammatory cytokines, leading to an acute phase response. Chronically elevated SAA concentrations are a prerequisite for the pathogenesis of secondary amyloidosis, a progressive and sometimes fatal disease characterized by the deposition in major organs of insoluble plaques composed principally of proteolytically cleaved SAA. This same process also may lead to atherosclerosis. There is a requirement for both positive and negative SAA control mechanisms to maintain homeostasis. These mechanisms permit the rapid induction of SAA expression to fulfill host-protective functions, but they also must ensure that SAA expression is rapidly returned to baseline levels to prevent amyloidosis. These mechanisms include modulation of promoter activity involving, for example, the inducer nuclear factor kB (NF-kB) and its inhibitor IkB, up-regulation of transcription factors of the nuclear factor for interleukin-6 (NF-IL6) family, and transcriptional repressors such as yin and yang 1 (YY1). Post-transcriptional modulation involving changes in MRNA stability and translation efficiency permit further up- and down-regulatory control of SAA protein synthesis to be achieved. In the later stages of the AP response, SAA expression is effectively down-regulated via the increased production of cytokine antagonists such as the interleukin-1 receptor antagonist (IL-1Ra) and of soluble cytokine receptors, resulting in less signal transduction driven by pro-inflammatory cytokines (Jensen and Whitehead, 1998).

The present invention provides methods and compositions of using agents that sequester and/or degrade amyloid or amyloid-like proteins and/or drusen, agents that prevent or reduce the production of such proteins and/or agents that prevent or reduce the toxic effects of such proteins for the treatment of ARMD to preserve vision.

The present inventor further postulates that agents that prevent or reduce the production and deposition of amyloid and amyloid-like proteins, and agents that sequester and/or degrade such proteins and agents that prevent such amyloid protein-induced cell death may be useful for protecting trabecular meshwork cells and other ocular cells in the anterior uvea and at the back of the eye, especially the retina and optic nerve head.

Compounds that may be useful for preventing the production of amyloid, amyloid-like proteins and/or drusen include: γ-secretase inhibitors such as talsaclidine (Hock et al. 2003), Xanomeline, L-689660, L-685458, McN-A-343, CDD-0097, fenchylamine, MG132, WPE-111-31C, MW-11-36C/26A, MW-167, CM-265, lactacystin, DNPS1 and DAPT (Lanz et al. 2003). Other compounds of use may include the statin family, e.g. pravastatin, atorvastatin (see Burns and Duff 2003) and presenilinase inhibitors such as pepstatin A (Xia 2003) and talsaclidine (Hock et al. 2003).

Compounds that may be useful for promoting degradation of amyloid, drusen and related proteins include glycoaminoglycans and congo red (J. Neurochem. 70: 292-298 [1998]).

Compounds that may be useful for promoting sequestration or clearance of amyloid, drusen and related proteins include gelsolin and ganglioside GM1 (Matsuoka et al. 2003). In addition, antibodies raised against drusen, and/or amyloid proteins and/or against amyloid-like proteins would be useful for sequestration and clearance of the former detrimental proteins as has been shown in the brain (Schenk et al. 1999; Janus et al. 2000; Morgan et al. 2000).

Compounds that may be useful for preventing or diminishing the neurotoxic effects of amyloid, drusen and related proteins include RS-0466 (Nakagami et al. 2002c; Nakagami et al. 2002b), V-type ATPase inhibitors (bafilomycin and concanamycin; Kane et al. 1999), tachykinin peptides and their non-peptide analogs (Yankner et al. 1990), α-lipoic acid (Zhang et al. 2001), propentofylline (Koriyama et al. 2003), glycogen synthase kinase-3β (GSK-3β) inhibitors (Eldar-Finkelman 2002; Caricasole et al. 2003), memantine (Frankiewicz and Parsons 1999), mixed cyclin-dependent kinase-GSK3β inhibitors (Damiens et al. 2001), COX-2 inhibitors (Xiang et al. 2002) and propentofylline (Koriyama et al. 2003).

The Compounds of this invention, can be incorporated into various types of ophthalmic formulations for delivery to the eye (e.g., topically, intracamerally, or via an implant). The Compounds are preferably incorporated into topical ophthalmic formulations for delivery to the eye. The Compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a Compound in a physiologically acceptable isotonic aqueous buffer. Further, the ophthalmic solution may include an ophthalmologically acceptable surfactant to assist in dissolving the Compound. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the Compound in a hydrophilic base prepared from the combination of, for example, carbopol-974, or the like, according to the published formulations for analogous ophthalmic preparations; preservatives and tonicity agents can be incorporated.

The Compounds are preferably formulated as topical ophthalmic suspensions or solutions, with a pH of about 4 to 8. The establishment of a specific dosage regimen for each individual is left to the discretion of the clinicians. The Compounds will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Formulation of Amyloid or Drusen Production Inhibitor for Topical Ocular Application

1% suspension of amyloid or drusen production inhibitor for topical ocular application

| Description | Conc. | | Units | Purpose |
|---|---|---|---|---|
| Amyloid or drusen | 1% | | W/V % | active ingredient |
| production inhibitor | | | | |
| hydroxypropyl | 0.5% | | W/V % | viscosity modifier |
| methylcellulose | | | | (2910) (E4M), USP |
| dibasic sodium | 0.2% | | W/V % | buffering agent |
| phosphate | | | | (anhydrous), usp |
| sodium chloride, usp | 0.75% | | W/V % | tonicity agent |
| disodium edta | 0.01% | | W/V % | chelating agent |
| (edetate disodium), | | | | |
| usp | | | | |
| polysorbate 80, nf | 0.05% | | W/V % | wetting agent |
| benzalkonium | 0.01% | | W/V % | preservative |
| chloride, nf | | | | |
| sodium | q.s. | pH | W/V % | pH adjust |
| hydroxide, nf | | | | |
| hydrochloric | q.s. | pH | W/V % | pH adjust |
| acid, nf | | | | |
| purified water, usp | q.s. | 100% | W/V % | vehicle |

In similar other examples, amyloid or drusen production inhibitor will be substituted by agents that sequester or degrade these proteins or agents that prevent the toxic effects of these proteins, such as those agents described above. All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, and the bibliography cited within these, to the extent that they provide exemplary procedural or other details supplementary to those set forth herein, are specifically incorporated herein by reference.

United States Patents

Books

Other Publications
Abler et al., RES. COMMUN. MOL. PATH. & PHARM. 92:177-189 (1996).
Asrani et al., INV. OPHTHALM. VIS. SCI. 38(13):2702-2710 (1997).
Asrani and Zeimer, BR. J. OPHTHALM. 79(8):776-770 (1995).
Bressler et al., SURVEY OF OPHTHALM. 32:375-413 (1988).
Brown, M., Gene therapy success for Alzheimer's?, DRUG DISCOV. TODAY 8:474-475 (2003).
Bums, M. and Duff, K., NEUROCHEM RES. 28:979-986 (2003).
Caricasole, A., et al. The Wnt pathway, cell-cycle activation and β-amyloid; novel therapeutic startegies in Alzheimer 's disease?, TRENDS PHARMACOL. SCI. 24:233-238 (2003).
Chabry J. et al. In vivo and in vitro neurotoxicity of the human prion protein (PrP) fragment P118-135 independently of the PrP expression, J. NEUROSCI. 23:462-469 (2003).
Ciulla et al., SURV. OPHTHALM. 43:134-146 (1998).
Curcio et al., INV. OPHTHALM. VIS. SCI. 37:1236-1249 (1996).
Damiens, E. et al., ONCOGENE 20:3786-3797 (2001).
Eldar-Finkelman, H., TRENDS MOL. MED. 8:126-132 (2002).
Fakforovich et al., NATURE 347:83-86 (1990).
Frankiewicz, T and Parsons, C. G., NEUROPHARMACOL. 38:1253-1259 (1999).
Ge-Zhi et al., TRANS. AM. OPHTHALM. SOC. 94:411-430 (1996).
Gragoudas et al., INV. OPHTHALM. VIS. SCI. 38(4):S17 (1997).
Hock, C. et al., Treatment with the selective muscarinic m1 agonist talsaclidine decreases cerebrospinalfluid levels of Aβ₄₂ in patients with Alzheimer's disease, AMYLOID: J. PROT. FOLD. DISORD. 10:1-6 (2003).
Husain et al., OPHTHALM. 104(8):1242-1250 (1997).
Janus, C. et al., Aβ peptide immunizaion reduces behavioural impairment and plaques in a model ofAlzheimer 's disease, NATURE 408:979-982 (2000).
Jen, L S, et al. Alzheimer's peptide kills cells of retina in vivo, NATURE 392:140-141 (1998).
Jensen L E and Whitehead A S, BIOCHEM. J. 334:489-503 (1998).
Jordat M S et al., PLANTA MED. 68:667-71 (2002).
Kane, M. D. et al., Inhibitors of V-type ATPases, bafilomycin A1 and concanamycin A, protect against β-amyloid-mediated effects on 3-(4,5-dimethylthiazol-2-yl)-2,5-diphenyltetrazolium bromide (MTT) reduction, J. NEUROCHEM. 72:1939-1947 (1999).
Koriyama, Y. et al., Propentofylline protects β-amyloid protein-induced apoptosis in cultured rat hippocampal neurons, EUR. J. PHARMACOL. 458:235-241 (2003).
Kumon, Y., Hosokawa, T., Suehiro, T., Ideda, Y., Sipe, J. D., and Hashimoto, K., Acute-phase, but not constitutive serum amyloid A (SAA) is chemotactic for cultured human aortic smooth muscle cells, AMYLOID 9:237-241 (2002a).
Kumon, Y., Suehiro, T., Faulkes, D. J., Hosakawa, T., Ideda, Y., Woo, P., Sipe, J. D., and Hashimoto, K., Transcriptional regulation of Serum Amyloid A1 gene expression in human aortic smooth muscle cells involves CCAAT/enhancer binding proteins (C/EBP) and is distinct from HepG2 cells, SCAND. J. IMMUNOL. 56:504-511 (2002b).
Kumon, Y., Suehiro, T., Hashimoto, K., and Sipe, J. D., Dexamethasone, but not IL-1 alone, upregulates acute-phase serum amyloid A gene expression and production by cultured human aortic smooth muscle cells, SCAND J. IMMUNOL. 53:7-12 (2001).
Lambert, M. P. et al., Diffusible, nonfibrillar ligands derivedform Aβ₁₋₄₂ are potent central nervous system neurotoxins, PROC. NAT. ACAD. SCI. USA: 95:6448-6453 (1998).
Liang, J. S., Sloane, J. A., Wells, J. M., Abraham, C. R., Fine, R. E., and Sipe, J. D., Evidence for local production of acute phase response apolipoprotein serum amyloid A in Alzheimer's disease brain, NEUROSCI. LETT. 225:73-76 (1997).
Lin et al., CURR. EYE RES. 13(7):513-522 (1994).
Liu, Y and Schubert, D., Cytotoxic amyloid peptodes inhibit cellular 3-(4,5-dimethylthiazol-2-yl)-2,5-diphenyltetrazolium bromide (MTT) reduction by enhancing MTT formazan exocytosis, J. NEUROCHEM. 69:2285-2293 (1997).
Lanz, T. A. et al., The γ-secretase inhibitor N-(N-(3,5-difluorophenacetyl)-L-alanyl]-S-phenylglycine t-butyl ester reduces Aβ levels in vivo in plsma and cerebrospinal fluid in young (plaque-free) and aged (plaque-bearing) Tg2576 mice, J. PHARMACOL EXPT. THER. 305:864-871 (2003).
LaVail et al., PROC. NAT'L ACAD. SCI. 89:11249-11253 (1992).
Marks, N. and Berg, M. J., APP processing enzymes (secretases) as therapeutic targets: insights from the use of transgenics (Tgs) and transfected cells, NEUROCHEM. RES. 28:1049-1062 (2003).
Matsuoka, Y. et al., Novel therapeutic approach for the treatment of Alzheimer's disease by peropheral administration of agents with an affinity for β-amyloid, J. NEUROSCI. 23:29-33 (2003). p0 Miida T., Yamada, T., Yamadera, T., Ozaki, K., Inano, K., Okada, M., Serum amyloid A protein generates pre-beta 1 high-density lipoprotein from alpha-migrating high-density lipoprotein, BIOCHEM. 38(51):16958-16962 (1999).
Morgan, D et al. Aβ peptide vaccination prevents memory loss in an animal model of Alzheimer's disease. NATURE, 408:982-985 (2000).
Naash et al., INV. OPHTHALM. VIS. SCI. 37:775-782 (1996).
Nakagami, Y and Oda, T. Glutamate exacerbates amyloid β1-42-induced impairment of long-term potentiation in rat hippocampal slices, JPN. J. PHARMACOL. 88:223-226 (2002a).
Nakagarni, Y. et al., A novel-sheet-breaker, RS-0406, reverses β-amyloid-induced cytotoxicity and impairment of long-term potentiation in vitro, BR. J. PHARMACOL. 137:676-682 (2002b).
Nakagami, Y. et al., EUR. J. PHARMACOL. 457: 11-17 (2002c).
Noell et al., INV. OPHTHALM. VIS. SCI. 5:450-472 (1966).
O'Hara, R., Murphy, E. P., Whitehead, A. S., FitzGerald, O., and Bresnihan, B., Acute-phase serum amyloid A production by rheumatoid arthritis synovial tissue, ARTHRITIS RES. 2:142-144 (2000).
Pike, C. J. et al., Neurodegeneration induced by β-amyloidpeptides in vitro: the role of peptide assembly state, J. NEUROSCI. 13:1676-1687 (1993).
Schenk, D. et al., Immunization with amyloid-β attenuates Alzheimer's-diesease-like pathology in the PDAPP mouse, NATURE 400:173-177 (1999).
Sickenberg et al., INV. OPHThALM. VIS. SCI. 38(4):S92 (1997).
Taylor et al., ARCH. OPHTHALM. 110:99-104 (1992).
Thomas et al., I0NV. OPHTHALM. VIS. SCI. 39(4):S242 (1998).
Thorn, C. F. and Whitehead, A. S., Differential glucocorticoid enhancement of the cytokine-driven transcriptional activation of the human actue phase serum amyloid A genes, SAA1 and SAA, J. IMMUNOL. 169:399-406 (2002).
Uhlar, C. M., and Whitehead, A. S., Serum amyloid A, the major vertebrate acute-phase reactant, EUR. J. BIOCHEM. 265:501-523 (1999).
Urieli-Shoval, S., Cohen, P., Eisenberg, S., and Matzner, Y., Widespread expression of serum amyloid A in histologically normal human tissue. Predominant localization to the epithelium, J. HISTOCHEM. CYTOCHEM. 46:1377-1384 (1998).
Xia, W. Relationship between presenilinase and γ-secretase, DRUG NEWS PERSPECT. 16:69-73 (2003)
Xiang, Z. et al., NEUROBIOL. AGING 23:327-334 (2002).
Yamazaki et al., BIOCHEMICAL AND BIOPHYSICAL RES. COMM., 290:1114-1122 (2002).
Yankner et al., SCIENCE 250:279-282 (1990).
Young, SURVEY OF OPHTHALMOL. 32:252-269 (1988).
Zhang, L. et al., α-Lipoic acid protects rat cortical neurons against cell death induced by amyloid and hydrogen peroxide through the Akt signaling pathway, NEUROSCI. LETT. 312:125-128 (2001)

I claim:

1. A method for treating AMD, said method comprising administering to a patient in need thereof a therapeutically effective amount of a composition comprising an agent that sequesters amyloid proteins in ocular tissue and/or an agent that degrades amyloid proteins in ocular tissue, wherein the sequestration and/or degradation modulates the expression of the amyloid proteins, such that the patient's condition is treated.

2. The method of claim 1, wherein said agent is a small organic molecule, antibody, protein, peptide, peptidomimetic, or nucleic acid.

3. The method of claim 1, wherein said agent is selected from the group consisting of

4. A method for treating AMD, said method comprising administering to a patient in need thereof a therapeutically amount of a composition comprising an agent that prevents the production of amyloid, amyloid-like proteins and/or drusen, said agent selected from the group consisting of y-secretase inhibitors, Xanomeline, L-689660, L-685458, McN-A-343, CDD-0097, fenchylamine, MG132, WPE-l11-31C, MW-11-36C/26A, MW-167, CM-265, lactacystin, DNPS1, DAPT, pravastatin, atorvastatin, presenilinase inhibitors and talsaclidine.

5. A method for treating AMD, said method comprising administering to a patient in need thereof a therapeutically effective amount of a composition comprising an agent that promotes degradation of amyloid, drusen and related proteins, said agent selected from the group consisting of glycoaminoglycans and congo red.

6. A method for treating AMD, said method comprising administering to a patient in need thereof a therapeutically effective amount of a composition comprising an agent that promotes sequestration or clearance of amyloid, drusen and related proteins, said agent selected from the group consisting of gelsolin, ganglioside GM1, and antibodies raised against drusen, and/or amyloid proteins and/or amyloid-like proteins.

7. A method for treating AMD, said method comprising administering to a patient in need thereof a therapeutically effective amount of a composition comprising and agent that prevent or diminish the neurotoxic effects of amyloid, drusen and related proteins, said agent selected from the group consisting of RS-0466, V-type ATPase inhibitors, tachykinin peptides and their non-peptide analogs, α-lipoic acid, propentofylline, glycogen synthase kinase-3β (GSK-3β) inhibitors, memantine, mixed cyclin-dependent kinase-GSK3β inhibitors, COX-2 inhibitors, and propentofylline.

8. A pharmaceutical composition comprising a therapeutically effective amount of an agent that sequesters amyloid proteins in ocular tissue and/or an agent that degrades amyloid proteins in ocular tissue and a pharmaceutical carrier.
